(19) United States
(12) Patent Application Publication
(10) Pub. No.: US 2026/0324966 A1
(43) Pub. Date: Oct. 22, 2026

(54) ROCKER SWITCH VALVE FOR BRONCHOSCOPE WITH RESPIRATORY SUPPORT AND METHODS OF USE THEREOF

(71) Applicant: Carilion Clinic, Roanoke, VA (US)

(72) Inventors: Edmundo R. Rubio, Roanoke, VA (US); David Ryan King, Roanoke, VA (US); Robert Fippinger, Roanoke, VA (US)

(21) Appl. No.: 19/650,489

(22) Filed: Apr. 17, 2026

Related U.S. Application Data
(60) Provisional application No. 63/791,019, filed on Apr. 18, 2025.

(51) Int. Cl. A61B 1/267; A61B 1/00; A61B 1/015; A61B 1/07

(52) U.S. Cl. CPC A61B 1/2676; A61B 1/00068; A61B 1/015; A61B 1/07

(57) ABSTRACT

A rocker-switch valve particularly useful with a bronchoscope is provided. The rocker-switch valve is capable of converting a bronchoscope that applies only suction to a bronchoscope that allows supply of oxygen in addition to vacuum. Also described are methods of using the bronchoscope device to restore a patient's oxygen saturation without removing the insertion tube.

### BACKGROUND

Bronchoscopy is a surgical technique that allows a doctor to view the airways of a patient. The bronchoscope typically includes an insertion tube that includes a suction channel and one or more flexible fiber optic instruments. The fiber optic instruments can include a light and a camera. The device is typically inserted through the patient's mouth or nasal cavity and allows the doctor to explore the patient's trachea, main stem bronchi, and some of the small bronchi. It is frequently used to remove small objects that have been inhaled, often by children. It is also often used to take biopsy samples of lesions found in a patient's lung.

As shown in FIG. 1, a bronchoscope 100 is being used on the lung 106 of a patient 102. The bronchoscope 100 includes a handle 102, an insertion tube 104, and a vacuum inlet 112. The tip 108 of the insertion tube extends into the small bronchi 110 of the patient’s lung 106. FIG. 2A shows a perspective view of a conventional non-disposable bronchoscopy device 200 that includes a disposable vacuum button assembly 210. The device has an insertion tube 220 and handle 230. An instrument port 240 is provided for inserting instruments through the insertion tube 220. FIG. 2B shows a front view of the bronchoscopy device. FIG. 2C shows a view of the disposable vacuum button assembly 210 described above. The disposable vacuum button assembly 210 includes a vacuum button 250, a vacuum tube 260, a valve body 280 and barbs 270 for holding a plastic vacuum line (not shown) onto the vacuum tube 260. During operation, the vacuum can be modulated by pressing button 250. After a surgical procedure, the disposable vacuum button assembly 210 can be removed from the bronchoscopy device 200 and discarded.

There is a need to improve the bronchoscope and methods of using the scope to make it more versatile and allow for procedures to be performed on patients experiencing desaturation of oxygen.

### SUMMARY OF THE INVENTION

In one aspect, the present invention relates to a rocker-switch valve that includes an oxygen inlet tube, a suction inlet tube, a rocker-switch valve body, a valve ball, and an actuator. The oxygen inlet tube may include oxygen inlet barbs on a distal end, the oxygen inlet barbs are adapted to interface with oxygen tubing. The suction inlet tube has suction inlet barbs on a distal end, the suction inlet barbs are adapted to interface with suction tubing. The rocker-switch valve body has an oxygen inlet port, a suction inlet port, and an outlet port. The oxygen inlet port is in fluid communication with the oxygen inlet tube, and the suction inlet port is in fluid communication with the suction inlet tube. The valve ball has a t-shape hole pattern. The actuator is configured to modify the position of the valve ball, wherein the actuator in a neutral position cuts off both the oxygen inlet port and suction inlet port from fluid communication with the outlet port, the actuator in a first position places the oxygen inlet port in fluid communication with the outlet port, and the actuator in a second position places the suction inlet port in fluid communication with the outlet port.

In one aspect, the rocker-switch valve includes an oxygen inlet with oxygen inlet barbs include a plurality of conical ridges that increase in diameter from the distal end of the oxygen inlet tube. The oxygen inlet barbs may have a plurality of conical ridges that increase in diameter from the distal end of the suction inlet tube. The oxygen inlet barbs may have successive increasing diameters of 6 mm, 7 mm, and 8 mm. The oxygen inlet has an internal diameter of about 5 mm, which is the same as the internal diameter of standard oxygen tubing. The suction inlet barbs may include a plurality of conical ridges that increase in diameter from the distal end of the suction inlet tube. The suction inlet barbs may have successive increasing diameters of 8 mm, 9 mm, 10 mm, and 11 mm. The suction inlet has an internal diameter of about 7 mm, which is the same as the internal diameter of standard suction tubing. It will be appreciated that the different internal diameters may be utilized in contexts where supply tubing has a different expected internal diameter.

In another aspect, the rocker-switch valve includes an actuator that has a paddle shape. The oxygen inlet tube and the suction inlet tube may form a U-shape each having an outward taper. The oxygen inlet tube and the suction inlet tube may have an outward taper forming an angle of 10°. The rocker-switch valve body further may also have two or more o-rings.

The invention also relates to methods of operating the bronchoscope device wherein the user of the bronchoscope can use the selector to cause either vacuum or pressurized gas to be applied within the fluid channel of the insertion tube of the bronchoscope. The method may entail delivering oxygen through the insertion tube when the patient's SpO₂ falls below 90%. This may allow continuing an operation whereby the insertion tube is not removed from the patient while the pressurized gas is being delivered. In addition, the oxygen may also be used to clear plugs/blocks at the distal end of the inlet tube that occur when objects for suction are too large for the lumen or to clear debris off of the fiber optic camera. This aspect can increase the efficiency of the device since it can reduce the amount of time procedures are paused. In one aspect, the bronchoscopy procedure is continued without removing the insertion tube after the patient's oxygen saturation is restored.

### BRIEF DESCRIPTION OF THE DRAWINGS

FIG. 1 shows a side view of a conventional bronchoscope delivered to a bronchos of the patient;

FIG. 2A shows a perspective view of a conventional non-disposable bronchoscope;

FIG. 2B shows a front view of a conventional non-disposable bronchoscope;

FIG. 2C shows a detail view of the vacuum button;

FIG. 3A shows a first perspective view of the rocker-switch shown in connection with the bronchoscope handle of FIG. 5.

FIG. 3B shows a second expanded perspective view of the rocker-switch shown in connection with the bronchoscope handle of FIG. 5.

FIG. 3C shows a top down view engineering drawing of the rocker-switch shown in connection with the bronchoscope handle of FIG. 5.

FIG. 3D shows a side view engineering drawing of the rocker-switch shown in connection with the bronchoscope handle of FIG. 5.

FIG. 3E shows a front view engineering drawing of the rocker-switch shown in connection with the bronchoscope handle of FIG. 5.

FIG. 4A shows a back view of a one-button rocker-switch bronchoscope handle according to an embodiment of the invention.

FIG. 4B shows a side view of the bronchoscope with the rocker-switch in the forward position.

FIG. 4C shows a side view of the bronchoscope with the rocker-switch in the backward position.

FIG. 4D shows a side view of the bronchoscope with the rocker-switch in the neutral position.

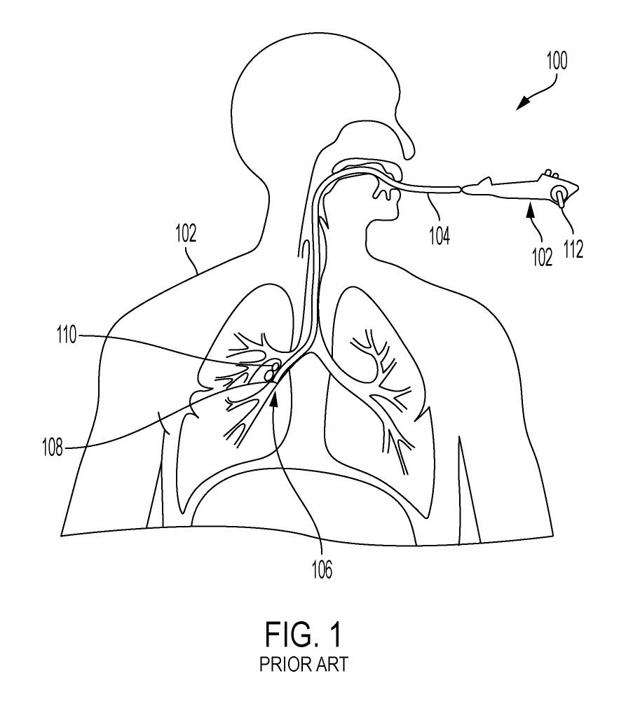

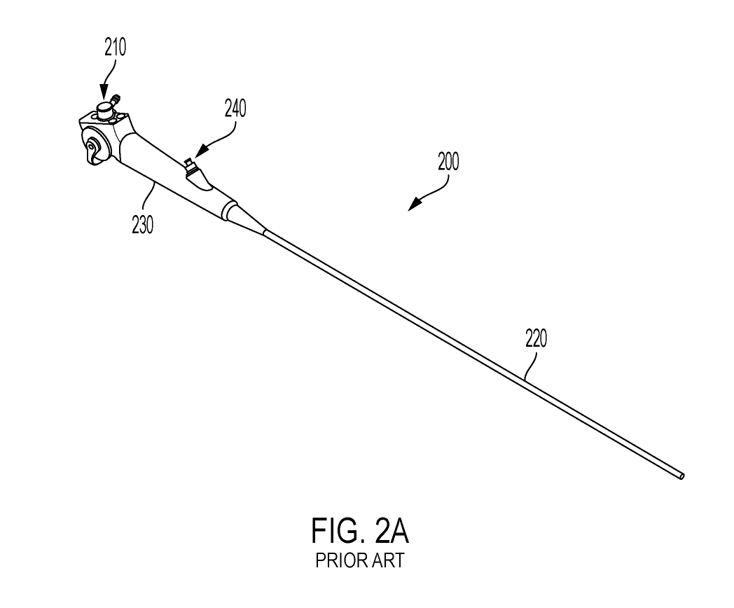

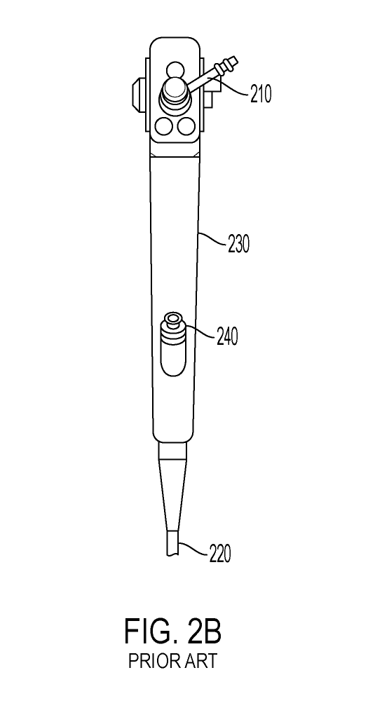

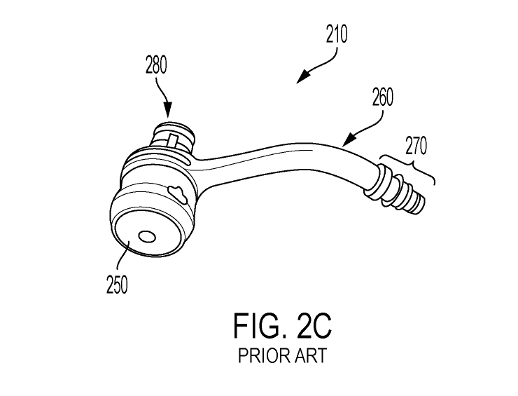

### DETAILED DESCRIPTION

The present inventors have found that there is a need for a treating physician to conveniently supply oxygen during the course of a bronchoscopy operations where a patient's oxygen saturation falls below 90% without removing the insertion tube of the bronchoscopy device. In addition, the oxygen may also be used to clear plugs/blocks at the distal end of the inlet tube that occur when objects for suction are too large for the lumen or to clear debris off of the fiber optic camera. This aspect can increase the efficiency of the device since it can reduce the amount of time procedures are paused. In the case of non-disposable bronchoscopes, the suction (e.g., vacuum) supply button is integrated with a valve having a suction inlet that includes barbs for connecting to a suction line. The suction button can then be replaced as necessary. The present inventors have made a rocker-switch valve that can be installed in place of the disposable suction supply button. The rocker-switch valve includes an oxygen inlet in addition to the suction inlet, and uses an actuator that may be in the shape of a paddle. The operator may alternatively switch on suction or oxygen supply by moving the paddle.

In one aspect, the present invention allows for placement of the gas inlets on the same side of the device (i.e., both point left or right giving a “U” shape instead of left and right giving a “T” shape). The inlets being on the same side of the device is important to the useability of the device because it allows oxygen and suction hoses to be positioned on one side of the patient. This avoid the problem of tubes drawing across the patient/provider and reducing the mobility and flexibility with which the provider can position and remove the device.

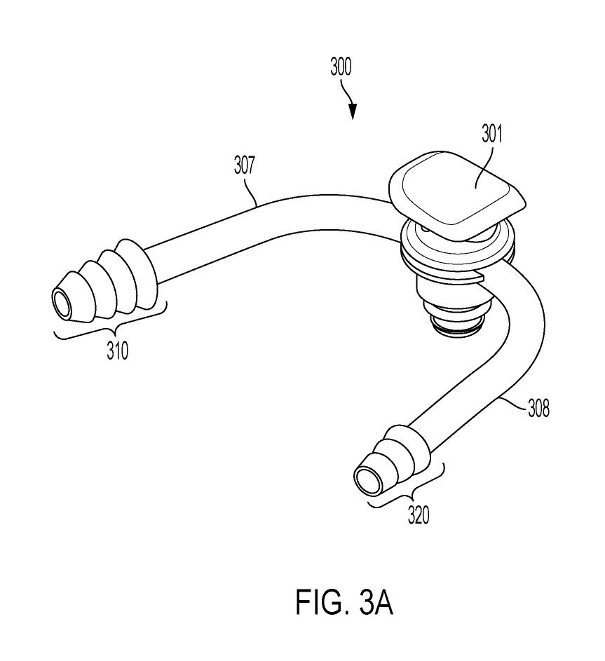

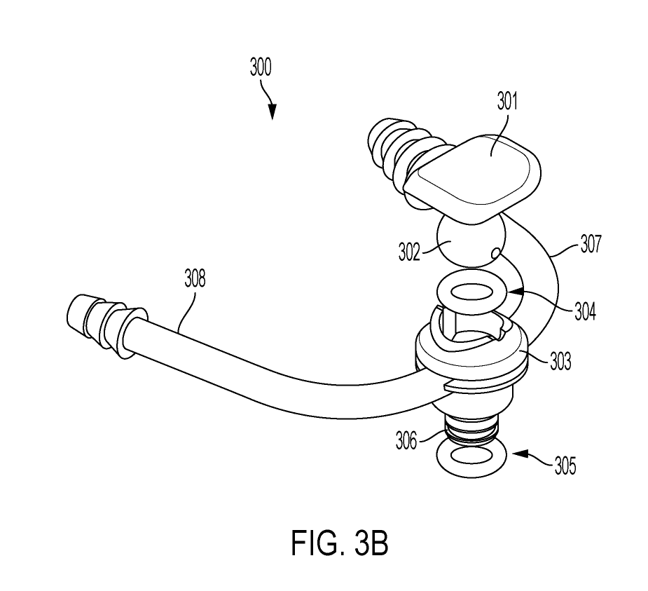

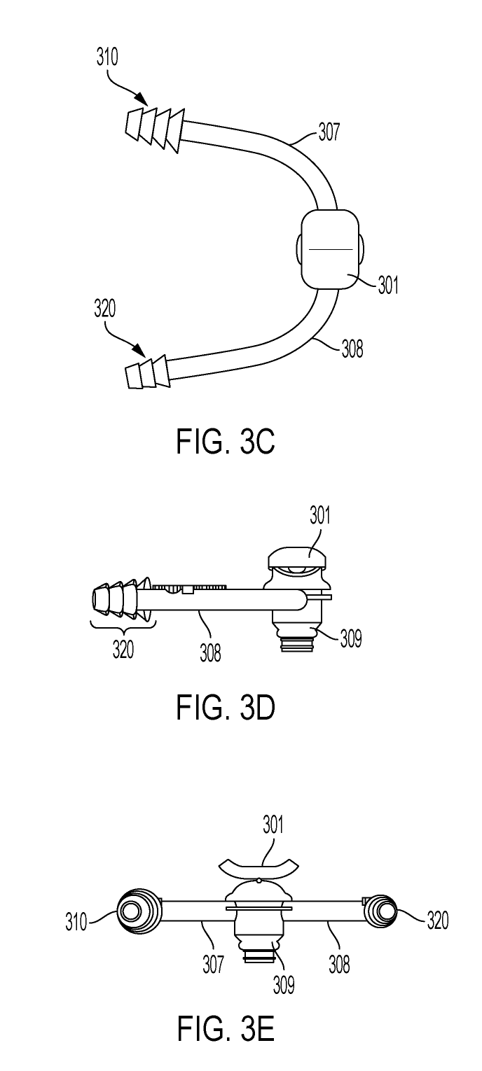

FIG. 3A shows a perspective view of the a rocker-switch valve 300. The rocker-switch valve 300 includes a suction inlet 307 for supplying vacuum to the bronchoscope, and a pressurized gas inlet 308 for supplying pressurized gas to the bronchoscope. The rocker-switch 300 includes a button 301 for toggling between vacuum and pressurized gas being supplied. The suction inlet 307 include barbs 310. The oxygen inlet 308 includes barbs 320. FIG. 3B shows an exploded view of the switch including the ball 302 and valve body 303. An outlet 306 connects the rocker-switch 300 to the bronchoscope handle (not shown). O-rings 304 and 305 are shown. FIGS. 3C through 3E show various views of the rocker-switch 300.

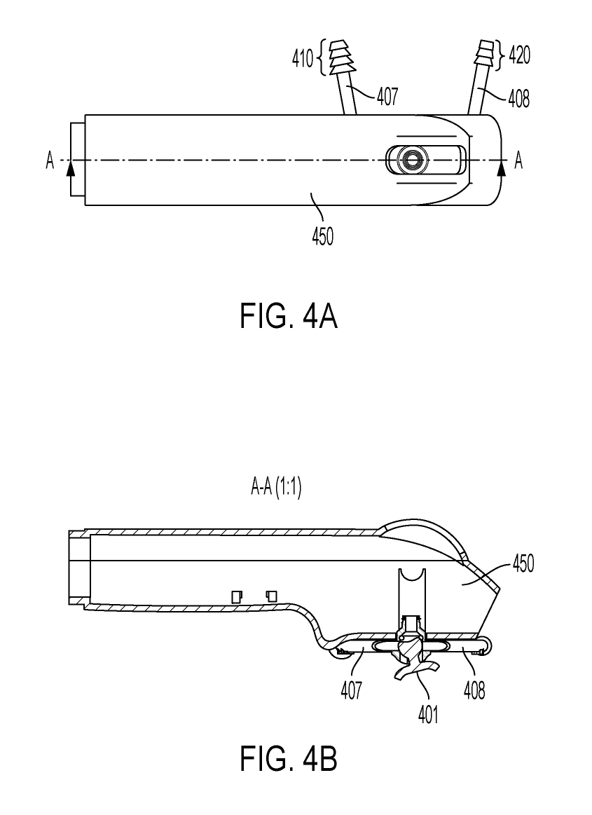

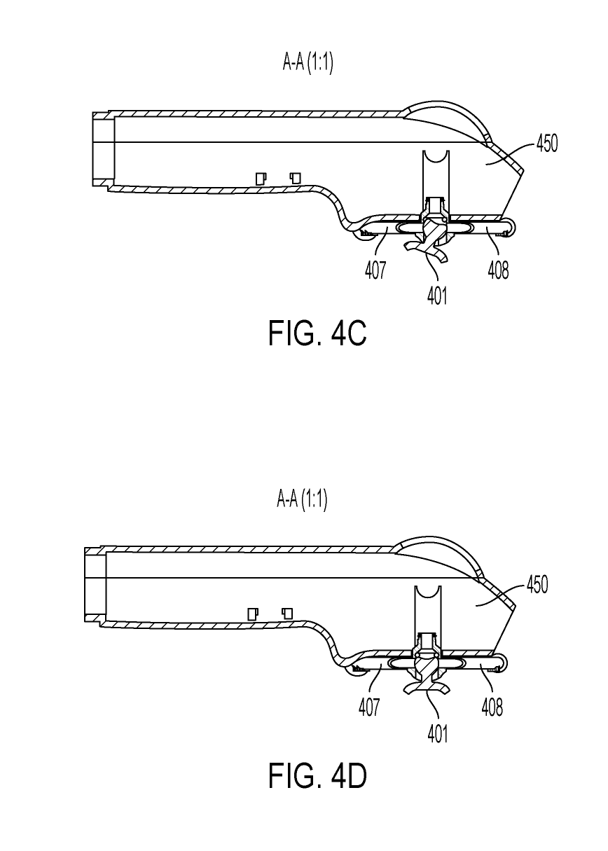

FIGS. 4A–4D show the functionality of the rocker-switch 401. FIG. 4A is a top-down view of the bronchoscope 400. The rocker-switch is integrated with a vacuum inlet 407 and pressurized gas inlet 408. FIG. 4B shows the rocker-switch 401 in the forward position. In the forward position, the pressurized gas is supplied to the insertion tube (not shown) of the bronchoscope. FIG. 4C shows the rocker-switch in the reverse position whereby a vacuum is supplied to the insertion tube. Finally, FIG. 4D shows the rocker-switch in the neutral position whereby neither vacuum nor pressurized gas is supplied to the insertion tube. The rocker-switch may be biased to remain in the neutral position when no forward or backward force is applied to the rocker-switch. Alternatively, the rocker-switch may be designed to stay in the position that is selected until the operator moves the rocker-switch back to the neutral position.

Also contemplated are methods of using the bronchoscope of the present invention during surgery. For example, the user of the bronchoscope can use the selector to cause either vacuum or pressurized gas to be applied within the fluid channel of the insertion tube of the bronchoscope during a surgical procedure. It is preferred that absent user intervention (pressing buttons or removing rocker-switch), neither vacuum nor pressurized gas is applied. During a procedures involving the bronchoscope, the vacuum or pressurized gas is off most of the time. The vacuum may be used to remove small debris when visualizing the patient via the insertion tube. This may be during a biopsy or during debris removal, especially for small children.

In one aspect, pressurized gas (e.g., oxygen) can be supplied to a patient whose SpO₂ falls below a specified level, for example 95%, 90%, 85% or 80%. This can allow the patient’s oxygen saturation to increase without removing the insertion tube. Alternatively, the pressurized gas can be used to clear debris from the end of the insertion tube and restore the field of view for an optical element, and allow the surgeon to better view the point of surgery. In a preferred aspect, the vacuum and pressurized gas inlets are on the same side of the bronchoscope. This can be beneficial for surgeons in that the bronchoscope can be more mobile than is the case when vacuum and pressurized gas tubes are on both sides of the device, obstructing movement within the operating room.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

What is claimed is:

1. A rocker-switch valve comprising:
an oxygen inlet tube, wherein the oxygen inlet tube has oxygen inlet barbs on a distal end, the oxygen inlet barbs are adapted to interface with oxygen tubing;
a suction inlet tube, wherein the suction inlet tube has a suction inlet barbs on a distal end, the suction inlet barbs are adapted to interface with suction tubing;
a rocker-switch valve body, the rocker-switch valve body comprising an oxygen inlet port, a suction inlet port, and an outlet port, wherein the oxygen inlet port is in fluid communication with the oxygen inlet tube, the suction inlet port is in fluid communication with the suction inlet tube;
a valve ball having a t-shape hole pattern; and
an actuator, wherein the actuator is configured to modify the position of the valve ball, wherein the actuator in a neutral position cuts off both the oxygen inlet port and suction inlet port from fluid communication with the outlet port, the actuator in a first position places the oxygen inlet port in fluid communication with the outlet port, and the actuator in a second position places the suction inlet port in fluid communication with the outlet port.

2. The rocker-switch valve of claim 1, wherein the oxygen inlet barbs include a plurality of conical ridges that increase in diameter from the distal end of the oxygen inlet tube.

3. The rocker-switch valve of claim 2, wherein the oxygen inlet barbs have successive increasing diameters of 6 mm, 7 mm, and 8 mm, and the oxygen inlet has an internal diameter of about 5 mm.

4. The rocker-switch valve of claim 1, wherein the suction inlet barbs include a plurality of conical ridges that increase in diameter from the distal end of the suction inlet tube.

5. The rocker-switch valve of claim 4, wherein the suction inlet barbs have successive increasing diameters of 8 mm, 9 mm, 10 mm, and 11 mm, and the suction inlet has an internal diameter of about 7 mm.

6. The rocker-switch valve of claim 1, wherein the actuator has a paddle shape.

7. The rocker-switch valve of claim 1, wherein the oxygen inlet tube and the suction inlet tube form a U-shape, optionally with each having an outward taper, and both the oxygen inlet tube and the suction inlet tube terminating on the same side of the device.

8. The rocker-switch valve of claim 7, wherein the outward taper of the oxygen inlet tube and the suction inlet tube forming an angle of 10°.

9. The rocker vale of claim 1, wherein the rocker-switch valve body further comprises two or more o-rings.

10. A bronchoscope device comprising:
an insertion tube, the insertion tube comprising a fluid channel and a fiber optic element;
a device handle connected to the insertion tube, the handle comprising:
a suction inlet tube, wherein the suction inlet tube has a suction inlet barbs on a distal end, the suction inlet barbs are adapted to interface with suction tubing having an internal diameter of about 7 mm;
a rocker-switch valve body, the rocker-switch valve body comprising an oxygen inlet port, a suction inlet port, and an outlet port, wherein the oxygen inlet port is in fluid communication with the oxygen inlet tube, the suction inlet port is in fluid communication with the suction inlet tube, the outlet port in fluid communication with the fluid channel of the insertion tube;
a valve ball having a t-shape hole pattern; and
an actuator, wherein the actuator is configured to modify the position of the valve ball, wherein the actuator in a neutral position cuts off both the oxygen inlet port and suction inlet port from fluid communication with the outlet port, the actuator in a first position places the oxygen inlet port in fluid communication with the outlet port, and the actuator in a second position places the suction inlet port in fluid communication with the outlet port.

11. The bronchoscope of claim 10, wherein the oxygen inlet barbs include a plurality of conical ridges that increase in diameter from the distal end of the oxygen inlet tube.

12. The bronchoscope of claim 11, wherein the oxygen inlet barbs have successive increasing diameters of 6 mm, 7 mm, and 8 mm.

13. The bronchoscope of claim 10, wherein the suction inlet barbs include a plurality of conical ridges that increase in diameter from the distal end of the suction inlet tube.

14. The bronchoscope of claim 13, wherein the suction inlet barbs have successive increasing diameters of 8 mm, 9 mm, 10 mm, and 11 mm.

15. The bronchoscope of claim 10, wherein the actuator has a paddle shape.

16. The bronchoscope of claim 10, wherein the oxygen inlet tube and the suction inlet tube form a U-shape, optionally with each having an outward taper, and both the oxygen inlet tube and the suction inlet tube terminating on the same side of the device.

17. The bronchoscope of claim 16, wherein the outward taper of the oxygen inlet tube and the suction inlet tube forming an angle of 10°.

18. The bronchoscope of claim 10, wherein the rocker-switch valve body further comprises two or more o-rings.

19. A method of conducting a bronchoscopy operation on a patient comprising inserting an insertion tube of a bronchoscope device into a patient, the bronchoscopy device comprising:
insertion tube, the insertion tube comprising a fluid channel and a fiber optic element;
a device handle connected to the insertion tube, the handle comprising:
an oxygen inlet tube, wherein the oxygen inlet tube has oxygen inlet barbs on a distal end, the oxygen inlet barbs are adapted to interface with oxygen tubing having an internal diameter of about 5 mm;
a rocker-switch valve body, the rocker-switch valve body comprising an oxygen inlet port, a suction inlet port, and an outlet port, wherein the oxygen inlet port is in fluid communication with the oxygen inlet tube, the suction inlet port is in fluid communication with the suction inlet tube, the outlet port in fluid communication with the fluid channel of the insertion tube;
a valve ball having a t-shape hole pattern; and
an actuator, wherein the actuator is configured to modify the position of the valve ball, wherein the actuator in a neutral position cuts off both the oxygen inlet port and suction inlet port from fluid communication with the outlet port, the actuator in a first position places the oxygen inlet port in fluid communication with the outlet port, and the actuator in a second position places the suction inlet port in fluid communication with the outlet port;
wherein the user of the bronchoscope can use the actuator to cause either suction or oxygen to be applied within the fluid channel of the insertion tube of the bronchoscope.

20. The method of claim 19, wherein the pressurized gas comprising oxygen is delivered through the insertion tube when the patient's SpO₂ falls below 90%.
